(19) United States
(12) Patent Application Publication
(10) Pub. No.: US 2012/0142596 A1
(43) Pub. Date: Jun. 7, 2012

(54) AGENT FOR STIMULATING MOBILIZATION OF ENDOTHELIAL PROGENITOR CELLS

(76) Inventors: Young Sook Son, Seoul (KR); Hyun Sook Hong, Seoul (KR)

(21) Appl. No.: 13/389,954

(22) PCT Filed: Aug. 12, 2010
(86) PCT No.: PCT/KR10/05307

§ 371 Date: Feb. 10, 2012

(30) Foreign Application Priority Data
Aug. 13, 2009 (KR) 10-2009-0074518

(51) Int. Cl. A61K 38/08; A61P 27/16; A61P 1/04; C07K 7/06; A61P 25/00

(52) U.S. Cl. 514/15.5; 530/327

(57) ABSTRACT

The present invention provides a composition for stimulating the mobilization of endothelial precursor cells, which comprises substance-P (SP). Substance-P according to the invention exhibits an activity of stimulating the mobilization of endothelial precursor cells from the bone marrow, thereby stimulating vasculogenesis. Thus, substance-P exhibits excellent effects on the prevention or treatment of diseases, including myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, and diabetic ulcer, which are accompanied by ischemic vascular injury or traumatic vascular injury.

### TECHNICAL FIELD

The present invention relates to an agent for stimulating mobilization of endothelial progenitor cells comprising substance-P.

### BACKGROUND ART

Ischemic vascular injury occurs because sufficient blood is not supplied due to vascular occlusion or vascular injury such that sufficient oxygen is not provided. This condition causes the massive necrosis of tissue cells, which threatens life. Typical examples thereof include myocardial infarction, angina, stroke, dementia and the like (Mymensingh Med J. 18, 264-72, 2009, Cardiol Rev, 16, 163-71, 2008). Thus, rapid regeneration of injured blood vessels is the most important step in the treatment of the disease.

It is known that the formation of new blood vessels occurs by angiogenesis and vasculogenesis. Angiogenesis is defined as the formation of new blood vessels by the sprouting of endothelial cells from pre-existing vessels, whereas vasculogenesis refers to the formation of new blood vessels by endothelial cells differentiated from endothelial progenitor cells (hereinafter also referred to as “EPCs”) that have mobilized to injury sites. If injury sites are small, vascular regeneration is possible by angiogenesis with the mobilization of the surrounding endothelial cells, but if injury sites are large, vascular regeneration is possible by vasculogenesis accompanied by the mobilization of EPCs from bone marrow or like.

EPCs are undifferentiated endothelial progenitor cells that can differentiate into mature endothelial cells. It is known that EPCs originate from bone marrow-derived hematopoetic cells, monocytic and nonmonocytic mononuclear cells, and tissue-resident cells, etc., and among these origins, bone marrow acts as a primary source (Stem Cell Rev, 3, 218-225, 2007).

It is known that EPCs present in the bone marrow are mobilized from the bone marrow to injury sites through various signal factors and participate in vascular regeneration. This role of EPCs is very important in acute ischemic vascular diseases. In myocardiac infarction (hereinafter referred to as “MI”), many studies on EPCs have been conducted. Badorff, C et al (2003) reported that, when EPCs were transplanted into an MI patient, the myocardial function of the patient was restored without side effects. Also, Yoon et al (2005) reported that, when EPCs were transplanted into an MI patient, the heart function of the patient was restored. Based on such results, it is expected that vascular repair or vasculogenesis would be promoted directly or indirectly by EPCs.

As the importance of the role of EPCs has increased, the mechanism in which EPCs mobilize from the bone marrow and home to injured sites has received increasing attention. G-CSF and GM-CSF which are currently clinically used were originally found as factors stimulating the proliferation and mobilization of neutrophils and macrophages, but since the effect of G-CSF on the mobilization of EPCs was recently found, G-CSF has been used as a releasing factor of therapeutic EPCs to peripheral blood. In addition, VEGF, MCP-1 (monocyte chemoattractant protein-1) and SDF-1 (stromal cell-derived factor-1) are factors known to regulate the mobilization of EPCs. Particularly, SDF-1 that is expressed by hypoxia was found to be involved in the mobilization of EPCs from the bone marrow and the homing of EPCs to injury sites in response to CXCR4 (Am J Physiol Cell Physiol. 292:C987-95, 2007, 2005). Also, there is a report that the simultaneous application of G-CSF and SDF-1 maximizes the effect of angiogenesis (Cardiovasc Res. 73, 823-832, 2007).

G-CSF is currently clinically used for vascular regeneration, but is known to cause many side effects. Examples of the side effects include pleocytosis, splenomegaly, medullary bone pain, nausea, vomiting, diarrhea, mucositis, neutropenic fever, fever, hair loss, weakness of the neuromuscular and skeletal systems, water retention, chest pain, loss of appetite, cheilitis, constipation, laryngopharyngitis, headache, eruption, difficult breathing, etc. Thus, there is an urgent need to develop substances specific for EPC mobilization, which do not cause side effects such as inflammation.

Substance-P (hereinafter also referred to as ‘SP’) has been known as a neurotransmitter that transmits pain to the central nervous system. It is a peptide consisting of 11 amino acids. It has the same amino acid sequence in humans, rats and rabbits, and thus is a kind of neurohormone that does not differentiate the species. Substance-P is expressed not only in nervous cells, but also in non-nervous tissues. It was found that substance-P is expressed in epithelial cells, endothelial cells (Watanabe M et al Jpn J Ophthalmol. 46, 616-20, 2002), macrophages, neutrophils (Ho W. Z. J. Immunol. 159, 5654-60 1997), cancer cells (Singh D et al. PNAS 97, 388-393, 2000), etc. Substance-P is a protein that binds to neurokinin receptors (NK-1, NK-2, and NK-3) on the cell surface to transmit a signal through G-protein-coupled receptor. This receptor is expressed in corneal epithelial cells (Watanabe M et al Jpn J Ophthalmol. 46, 616-20, 2002), skin keratinocytes (Leu J Y et al. Br J. Dermatol. 155, 657-62 2006), mesenchymal stem cells, etc. In view of this expression and action in various tissues, substance-P is expected to play an important role in nervous-immune system interactions, myelofibrosis, cancer cell proliferation and the like in addition to pain transmission which is the previously known function.

The action of substance-P on mesenchymal stem cells was reported in the study of Pranela Rameshwar (2001). The report suggests that substance-P plays a positive role in the support of hematopoietic cells by mesenchymal stem cells. Namely, it was found that substance-P can also be involved in immune system regulation, because it stimulate mesenchymal stem cells to secrete various cytokines and growth factors, thereby promoting hematopoiesis (Pranela Rameshwar. et al. Journal of Neuroimmunology 121, 22-31, 2001).

It was found that, when substance-P is used together with IGF or EGF, it stimulates the mobilization of the surrounding epithelial cells to promote corneal regeneration (Nakamura M. et al. Diabetologia. 46, 839-42, 2003 Yamada N et al. Invest Ophthalmol V is Sci. 45, 1125-31, 2004). Based on this effect of substance-P on the stimulation of mobilization of corneal epithelial cells, Santen Pharmaceutical Co., Ltd. (Japan) is conducting the development of eye-drops containing substance-P. Also, Rook J M (2007) confirmed that, when a mixture of intrasite gel and substance-P is applied to a wound, the healing of the wound is promoted.

Although various uses and expected effects of substance-P are increasing, these effects are mostly attainable when substance-P is locally applied, and the effects are exhibited through the stimulation of the surrounding tissue cells. Thus, such use of substance-P is effective for small injuries, but in the case of large injuries, healing is delayed or cannot occur. Particularly, in the case of wounds and ulcers which are difficult to heal, tissue repair is delayed, because the distribution of blood vessels is poor, and sustained inflammatory reactions, infections and the like are accompanied. To heal such diseases, vascular repair should be promoted. Thus, it is impossible to heal such diseases only by the stimulation of cells surrounding the injury, and the introduction of EPCs from blood should be accompanied (Gibran N. S. et al Journal of Surgical Research 108, 122-128, 2002).

Although many roles of substance-P have been reported to date, the function of substance-P associated with EPCs has not yet reported. Accordingly, if substance-P has a function specific for EPC mobilization, substance-P is expected to play an important role as a vascular regeneration promoter in various diseases accompanied by ischemic vascular injury. Thus, the present inventors have conducted extensive studies, thereby completing the present invention.

### DISCLOSURE

### Technical Problem

The present invention is to provide a composition or agent for mobilizing endothelial progenitor cells from the bone marrow to injured vascular sites to stimulate vasculogenesis and prevent or treat vascular injury-related diseases.

### Technical Solution

The present invention provides a composition for stimulating mobilization of endothelial progenitor cells, which comprises substance-P as an active ingredient.

The present invention also provides a composition for stimulating mobilization of endothelial progenitor cells into blood, which comprises substance-P as an active ingredient.

The present invention also provides a composition for stimulating vasculogenesis, which comprises substance-P as an active ingredient.

The present invention also provides a composition for stimulating vasculogenesis by mobilization of endothelial progenitor cells, which comprises substance-P as an active ingredient.

The present invention also provides a composition for stimulating vasculogenesisby stimulating the mobilization of endothelial progenitor cells from bone marrow into blood, wherein the composition comprising substance-P as an active ingredient.

The present invention also provides a composition for preventing or treating ischemic vascular injury or traumatic vascular injury, which comprises substance-P as an active ingredient.

The present invention also provides a composition for preventing or treating myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, or diabetic ulcer.

The composition of the present invention is a composition for intra-bone marrow administration, intravenous administration, subcutaneous administration or intraperitoneal administration.

The composition of the present invention may comprise, in addition to the active ingredient substance-P, pharmaceutical suitable, physiologically acceptable adjuvants. Examples of such adjuvants include excipients, disintegrants, sweetening agents, binders, coating agents, blowing agents, lubricants, and flavoring agents.

The composition of the present invention may be formulated as a pharmaceutical composition comprising one or more pharmaceutically acceptable carriers for administration, in addition to the active ingredient as described above. Examples of such formulations include granules, powders, tablets, coated tablets, capsules, syrup, juice, suspensions, emulsions, ointments, cream, gel, drops, aerosol, injectable liquid, etc. For example, for formulation in the form of tablets or capsules, the active ingredient may be combined with any oral nontoxic pharmaceutically acceptable carrier such as ethanol, glycerol, water and the like. Moreover, when desired or necessary, suitable binders, lubricants, disintegrating agents and coloring agents can also be incorporated into the mixture. Suitable binders include, but are not limited to, starch, gelatin, natural sugars such as glucose or beta-lactose, corn sweeteners, natural and synthetic gums such as gum acacia, tragacanth gum or sodium oleate, sodium stearate, magnesium stearate, sodium benzoate, sodium acetate, sodium chloride, etc. Suitable disintegrants include, but are not limited to, starch, methyl cellulose, agar, bentonite, xanthan gum, etc.

Examples of pharmaceutically acceptable carriers, which can be used to formulate the inventive composition in the form of liquid solutions, include saline solution, sterile water, Ringer's solution, buffered saline solution, albumin injectable solution, dextrose solution, maltodextrin solution, glycerol, ethanol, and mixtures of two or more thereof. If necessary, the inventive composition may also contain other conventional additives, such as antioxidants, buffers and bacteriostatic agents. Moreover, the inventive composition may additionally contain diluents, dispersants, surfactants, binders and lubricants in order to formulate it into injection formulations, such as aqueous solutions, suspensions and emulsions, pills, capsules, granules and tablets. Furthermore, it may preferably be formulated depending on particular diseases orits components, using the method described in Remington's Pharmaceutical Science, Mack Publishing Company, Easton Pa., which is a suitable method in the relevant field of art.

The present invention also provides the uses of the composition, which contains substance-P as an active ingredient, for stimulating vasculogenesis, stimulating mobilization of endothelial progenitor cells, stimulating vasculogenesis by mobilization of endothelial progenitor cells, stimulating the mobilization of endothelial progenitor cells from bone marrow into blood, and stimulating vasculogenesis by stimulation of mobilization of endothelial progenitor cells.

The present invention also provides the use of the composition, which contains substance-P as an active ingredient, for preventing or treating ischemic vascular injury or traumatic vascular injury.

The present invention also provides the use of the composition, which contains substance-P as an active ingredient, for preventing or treating myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, or diabetic ulcer.

The present invention also provides a method for treating vasculogenesis-related diseases, preferably myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, or diabetic ulcer, the method comprising administering a therapeutically effective amount of substance-P to a mammal.

As used herein, the term “therapeutically effective amount” refers to that amount of an active ingredient or pharmaceutical composition that will elicit the biological or medical response of an animal or human that is being sought by a researcher, veterinarian, medical doctor or clinician, and encompasses an amount of the active ingredient or pharmaceutical composition which will ameliorate the symptoms of the disease or disorder being treated. As apparent to those skilled in the art, the therapeutically effective dose and administration times of the active ingredient in accordance with the present invention may vary depending upon desired therapeutic effects. Therefore, an optimal dose of the active ingredient to be administered can be easily determined by those skilled in the art. For example, an effective dose of the active ingredient is determined taking into consideration various factors such as kinds of disease, severity of disease, contents of active ingredients and other components contained in the composition, kinds of formulations, age, weight, general health status, sex and dietary habits of patients, administration times and routes, release rates of the composition, treatment duration, and co-administered drugs. In the inventive method for treating vasculogenesis-related diseases, preferably myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, or diabetic ulcer, substance-P is administered at a dose of 0.001-0.5 mg/day, and preferably 0.0001-0.005 mg/kg, once a day for adults.

The composition of the present invention may be administered via various routes, including oral, sublingual, intrarectal, transdermal, subcutaneous, intramuscular, intraperitoneal, intravenous, intraarterial, intrathecal, and intra-bone marrow routes.

The present invention also provides an agent for stimulating mobilization of endothelial progenitor cells, which comprises substance-P in an amount effective for inducing mobilization of endothelial progenitor cells.

The inventive agent for stimulating mobilization of endothelial progenitor cells stimulates the mobilization of endothelial progenitor cells from bone marrow into blood. Endothelial progenitor cells that mobilized from bone marrow into blood by substance-P mobilize to injured vascular sites and participate in vasculogenesis.

The fact that substance-P stimulates the mobilization of endothelial progenitor cells from bone marrow was found first by the present inventors.

The inventive agent for stimulating mobilization of endothelial precursor cells can be used for the prevention or treatment of ischemic vascular injury or traumatic vascular injury. Thus, it can be used for the prevention or treatment of myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, or diabetic ulcer.

The formulation, dose, administration route and the like of the inventive agent for stimulating mobilization of endothelial progenitor cells can be determined within a conventional range known in the art. For these, reference may be made to those described in the specification with respect to the composition comprising substance-P.

The present invention also provides a method for stimulating mobilization of endothelial progenitor cells, the method comprising administering to a subject substance-P in an amount effective for inducing mobilization of endothelial progenitor cells.

When substance-P is administered to the subject, endothelial progenitor cells in bone marrow mobilize from the bone marrow to blood, preferably, an injured blood vessel, to stimulate vasculogenesis. Substance-P may be adapted for administration by any convenient route conventional in the art. Preferably, it may be administered intra-bone marrow, intravenously, subcutaneously or intrapertioneally, and more preferably, it may be administered intravenously. Even when substance-P of the present invention is administered directly into the bone marrow or it is administered intravenously; it shows excellent ability to stimulate mobilization of endothelial progenitor cells.

The subject to which the method for stimulating mobilization of endothelial precursor cells is applied is preferably a patient having, or risk of, at least one disease selected from among is chemicvascular damage or traumatic vascular injury, preferably myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, and diabetic ulcer.

The present invention also provides a method for stimulating vasculogenesis in the injured vascular tissue of a subject, comprising the steps of: (a) administering to a subject substance-P in an amount effective for inducing mobilization of endothelial progenitor cells from bone marrow, thereby stimulating the mobilization of endothelial progenitor cells from bone marrow to blood; (b) collecting endothelial progenitor cells from the blood; and (c) introducing the collected endothelial progenitor cells into the subject.

In the step (a) of the above method, substance-P may be administered by any conventional route. Preferably, it may be administered intra-bone marrow, intravenously, subcutaneously or intraperitoneally, and more preferably, it may be administered intravenously. Unlike other stimulators of mobilization of bone marrow-derived endothelial progenitor cells, for example, G-CSF, substance-P sufficiently stimulates the mobilization of endothelial progenitor cells to blood, even when it is administered intravenously as well as intra-bone marrow. Thus, it reduces difficulty in collection of bone marrow-derived endothelial progenitor cells.

In the step (b), the blood is blood obtained from any part of the subject, preferably peripheral blood obtained from the subject, and the collection of the endothelial progenitor cells from the blood may be carried out by any conventional method known in the art. For example, the endothelial progenitor cells can be obtained by isolating mononuclear cells from peripheral blood, placing the isolated cells in a fibronectin-coated dish, and culturing the cells in EGM (endothelial growth medium) to selectively proliferate only EPCs.

In the step (c), the introduction of the collected endothelial progenitor cells into the subject may be performed by any conventional method known in the art. For example, the collected endothelial progenitor cells may be applied directly to an injury site in a gel or solution form (hydrogel, fibrin glue or collagen) or may be administered by various routes such as intravenous or subcutaneous routes.

### Advantageous Effects

The composition comprising substance-P in accordance with the present invention exhibits the effects of stimulating mobilization of endothelial progenitor cells, and stimulating vasculogenesis by the mobilization of endothelial progenitor cells. The composition according to the present invention exhibits the effect of preventing or treating diseases, including myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, and diabetic ulcer.

### DESCRIPTION OF DRAWINGS

FIG. 1 is a graph showing the effect of intravenous administration of SP on the stimulation of EPC mobilization.

FIG. 2 is a set of photographs showing the results of examining the presence of EPCs in peripheral blood, obtained after administration of SP, using EPC markers (CD34 and UAE-1).

FIG. 3 is a set of photographs showing the tube-forming ability of cells collected from peripheral blood obtained after administration of SP.

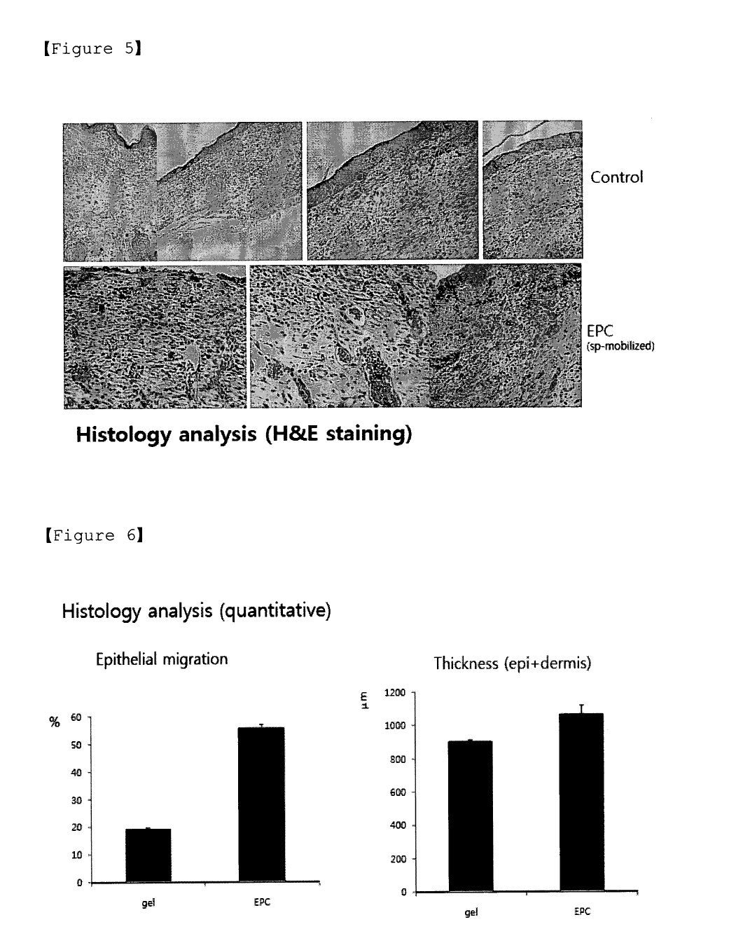

FIG. 4 is a set of photograph showing the results of applying EPCs to a mouse skin wound.

FIG. 5 is a set of photographs showing the cross-section of a mouse wound applied with EPC.

FIG. 6 is a set of graphs showing the results of quantifying the epidermal and dermal changes of a mouse skin wound, applied with EPCs, using a microscope.

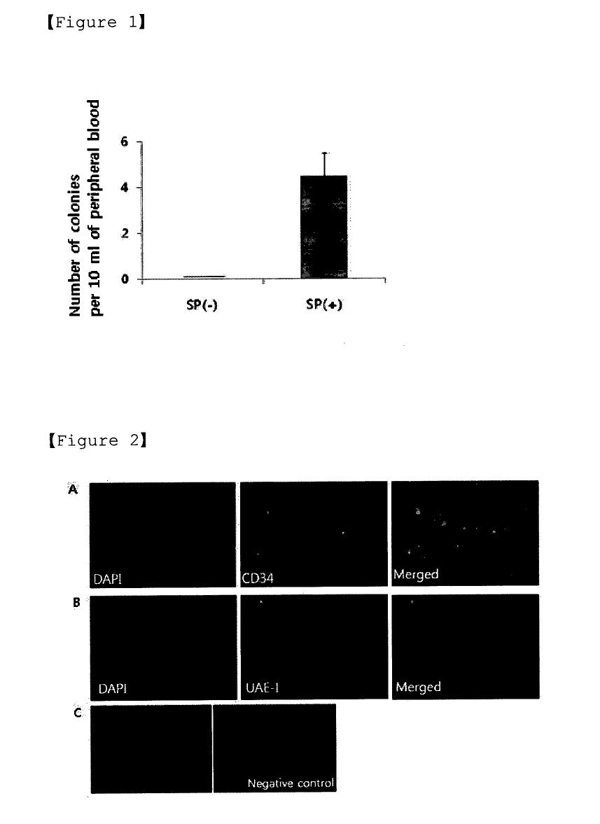

FIG. 1 is a graph showing the effect of intramedullary administration of SP on the stimulation of EPC mobilization.

FIG. 8 is a graph showing the amount of EPCs in peripheral blood after intravenous injection of each of SP and G-CSF.

FIG. 9 is a set of graphs showing the amounts of neutrophils, monocytes and lymphocytes after intravenous injection of each of SP and G-CSF.

FIG. 10 is a graph showing the amount of EPCs in peripheral blood obtained after intra-bone marrow injection of each of SP and G-CSF.

FIG. 11 is a set of graphs showing the amounts of neutrophil, monocyte, lymphocyte and eosinophil in peripheral blood after intra-bone marrow injection of each of SP and G-CSF.

### MODE FOR INVENTION

Advantages and features of the present invention, and a method for achieving them will be clarified with reference to examples that will be described later. The present invention may, however, be embodied in different forms and should not be construed as limited to the examples set forth herein. Rather, these examples are provided so that the disclosure of the present invention will be thorough and complete, and will fully convey the scope of the present invention to those skilled in the art. The scope of the present invention should be defined by the appended claims.

### EXAMPLES

### Example 1

### Examination of Effect of Intravenous Administration of SP on Stimulation of EPC Mobilization

(1) Intravenous Administration of SP

Ketamine hydrochloride (50 mg/ml Yuhan, Korea) and Rompun (xylazine hydrochloride, 23.32 mg/ml Bayer Korea) were mixed at a ratio of 2:1, and then 900 μl of the mixture (600 μl of ketamine hydrochloride and 300 μl of Rompun) was injected intramuscularly into New Zealand white rabbits (male, 2.5 kg, n=3). 10 minutes after the intramuscular injection, the same anesthetic (200 μl of ketamine hydrochloride and 100 μl of Rompun) was injected intravenously into the ear vein of each rabbit to completely anesthetize the rabbits. Then, 5 nmole/kg of SP was dissolved in physiological saline solution, and 1 ml of the solution was administered slowly into the ear vein of one rabbit group. As a control, 1 ml of 1×PBS was administered into the ear vein of another rabbit group. 24 hours after the administration, the same amount of each of SP and 1×PBS was injected once more.

(2) Collection of Peripheral Blood

3 days after the first injection, peripheral blood was collected from the ear blood vessels of the rabbits, and 10 ml of the collected blood was two-fold diluted in PBS, and then carefully placed on Ficoll contained in a tube (Ficoll: peripheral blood=1:1 v/v). Next, the blood dilution was centrifuged at 2200 rpm for 25 minutes. As a result, a red blood cell (RBC) layer, a ficoll layer, a mononuclear cell layer and a plasma layer, from bottom to top, were formed. The uppermost plasma layer was removed and only the mononuclear cell layer was separated from the tube and transferred into a new tube. 30 ml of PBS was added to the obtained mononuclear cell layer and centrifuged at 1500 rpm for 5 minutes (washing step). After the centrifugation, the precipitated cell pellet was suspended in EGM-2 (endothelial growth medium-2, Lonza).

(3) Selective Culture of EPCs

The cells suspended in the medium were seeded in a 100-mm dish (Falcon) coated with fibronectin and were cultured in EGM-2 (endothelial growth medium-2, Lonza) for 2 weeks (5% CO₂ and 37° C.). After the culture, the produced colonies were counted, and the count results are shown in FIG. 1. As shown in FIG. 1, in the peripheral blood of the control group administered with PBS, EPCs were not substantially found, whereas, in the peripheral blood of the test group intravenously administered with SP, the amount of EPCs significantly increased. This demonstrated the effect of SP on the stimulation of EPC mobilization.

(4) Examination of Isolated and Cultured Cells

1) Immunohistochemical Staining

In order to examine the presence of EPCs in peripheral blood using the EPC markers CD34 and UEA, EPCs (2×10⁴ cells/well) that have been isolated and cultured from the above blood were incubated on cover slips for CD34 test and UEA test, respectively. After incubation in a 5% CO₂ incubator at 37° C. for 24 hours, the cells were washed with PBS. Then, the cells were fixed with 3.7% paraformaldehyde and washed again with PBS.

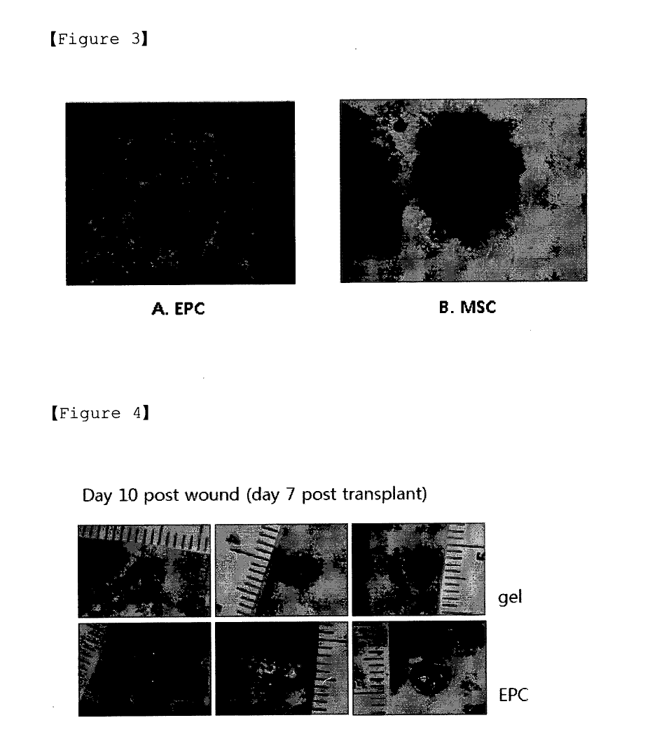

The washed cells were blocked with 20% goat serum and treated with anti-CD 34 antibody at room temperature for 1 hour. Then, the cells were washed again with PBS and treated with FITC-conjugated anti-mouse IgG at room temperature for 1 hour. To remove excess of antibodies, the cells were washed with PBS, counterstained with DAPI, and then mounted. After mounting, the expression of EPC-specific marker was observed with a fluorescence microscope (Leica). The observation results are shown in FIG. 2A.

The washed cells were blocked with 20% goat serum and treated with UEA-biotin at room temperature for 1 hour. Then, the cells were washed again with PBS and treated with streptavidin-FITC at room temperature for 1 hour. The cells were washed with PBS, counterstained with DAPI, and then mounted. After mounting, the expression of the cells was observed with a fluorescence microscope (Leica). The observation results are shown in FIG. 2B.

For negative control in immune fluorescence staining, cultured cells were treated with only secondary antibody, and the expression thereof was observed (FIG. 2C).

As can be seen in FIG. 2, the peripheral blood obtained after intravenous administration of SP contained a large amount of EPCs.

2) Examination of Ability to Form Tubes In Vitro

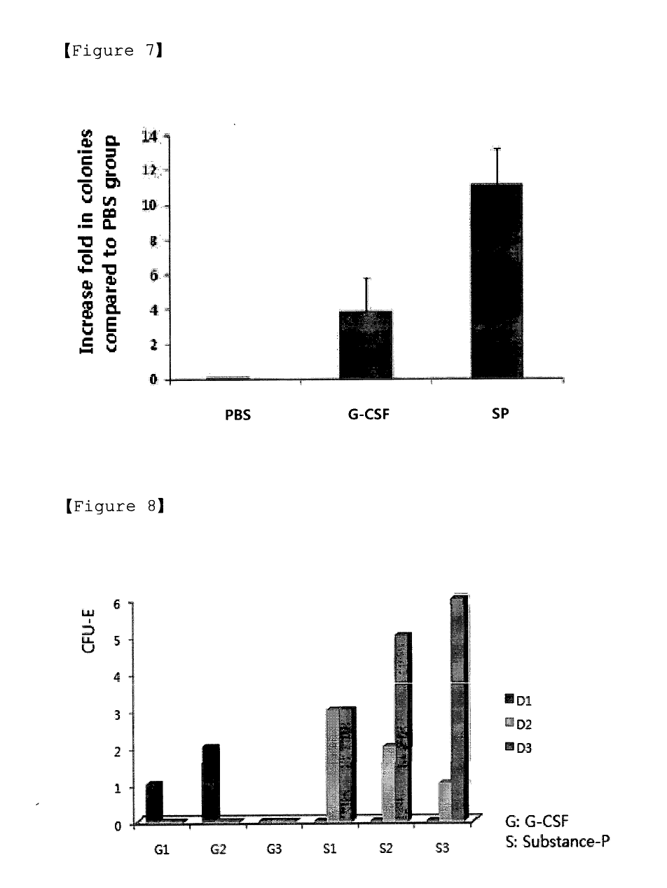

In order to examine the blood vessel-forming ability of the EPCs that have been isolated and cultured from the blood, the cells were cultured on Matrigel (BD). For this purpose, 200 μl of Matrigel was added to each well of a 96-well plate and incubated at 37° C. for 30 minutes. The cells were added to each well containing Matrigel at a density of 2×10⁴ and cultured for 12 hours. Then, whether tubes were formed was observed with a microscope, and the observation results are shown in FIG. 4A. When mesenchymal stem cells (MSCs) were cultured in the same manner as described above, colonies were formed as shown in FIG. 4B. It could be seen that EPCs contained in the peripheral blood obtained after intravenous administration of SP had the ability to form blood vessels.

3) Examination of Ability to Form Tubes In Vivo

Nude balb/c mice were prepared (three mice per group). The skin of the animals was wounded by a 8-mm biopsy punch, and the epidermis and the dermis were all removed. 3 days after wounding, prepared EPCs were transplanted into the animals at a density of 1×10⁶ cells per animal.

After the cells have been prepared, they were labeled with a fluorescent substance. The cells were suspended in hydrogel and thoroughly mixed. The hydrogel is a hydrogel form containing poloxamer or collagen, and when it is transplanted in vivo, it hardens to form a semi-solid. The cells mixed with the hydrogel were transplanted into the wound site.

A control group was applied only with hydrogel without cells.

Dressing was done with Mepitel and Tegaderm. One week after the transplantation, the mice were sacrificed, and skin samples were isolated from the animals.

In order to examine the degree of wound repair and the ability to form blood vessels, the skin tissue was sectioned in paraffin, and the sections were stained with hematoxylin and eosin. After the staining, vascular formation was observed, and the regeneration of epidermal and dermal tissues was quantified using image J program.

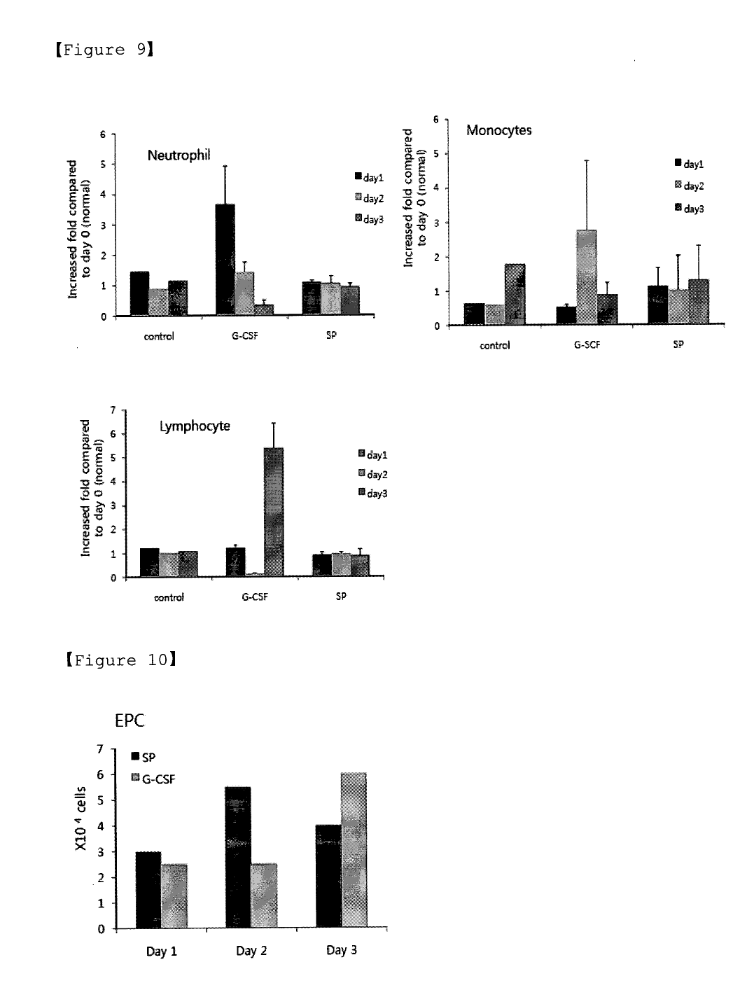

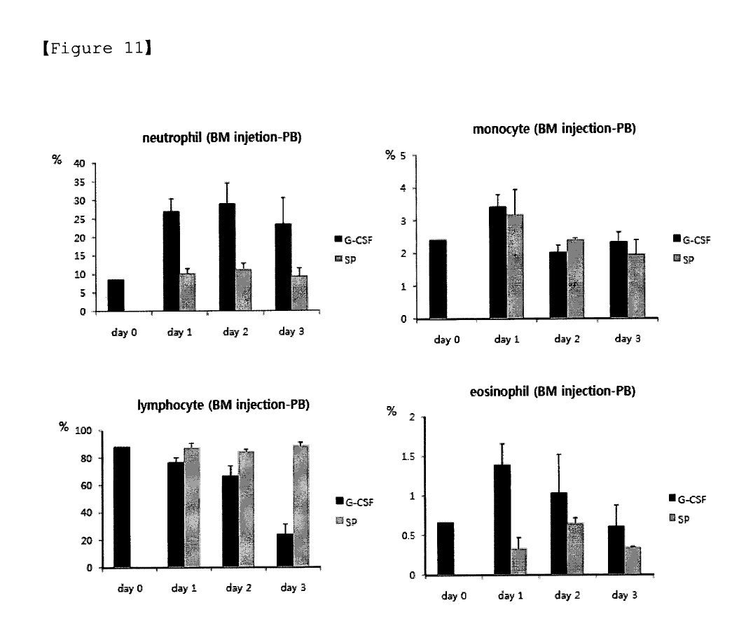

The results are shown in FIGS. 4 to 6. FIG. 4 is a photograph showing the mouse skin wound, and FIG. 5 is a photograph showing the cross-section of the skin tissue of the wound site. As shown in FIG. 5, in the control group, no blood vessel was formed, but in the skin tissue of the mice transplanted with the cultured EPCs that have been obtained from the peripheral blood after intravenous administration of SP, blood vessels were formed. Also, as shown in FIG. 6, epithelial mobilization significantly increased, and the epidermal and the dermis were more rapidly regenerated. Namely, it was found that many blood vessels were formed in the dermal portion of the group administered with the EPCs cultured from the peripheral blood collected from the mice administered with SP, and the regeneration of epidermis and dermis was accelerated.

### Example 2

### Examination of Effect of Intra-Bone Marrow Administration of SP on Stimulation of EPC Mobilization

(1) Examination of Bone Marrow Aspirate after Administration of SP

1) Intra-Bone Marrow Administration of SP

Ketamine hydrochloride (50 mg/ml Yuhan, Korea) and Rompun (xylazine hydrochloride, 23.32 mg/ml Bayer Korea) were mixed at a ratio of 2:1, and then 900 μl of the mixture (600 μl of ketamine hydrochloride and 300 μl of Rompun) was injected intramuscularly into New Zealand white rabbits (male, 2.5 kg, n=4). 5 minutes after the intramuscular injection, the same anesthetic (200 μl of ketamine hydrochloride and 100 μl of Rompun) was injected intravenously into the ear of each rabbit to completely anesthetize the rabbits.

SP (Sigma, 4 nmole/kg in saline) was injected into the right iliac crest of one rabbit group at an injection volume of 0.1 ml using a 18G spinal needle.

As a negative control, 1×PBS was injected into the left iliac crest of another rabbit group at an injection volume of 0.1 ml using a 18G spinal needle. As a positive control, G-CSF (CJ Pharma Leukokine Inj. 150; 2.5 μg/kg) was injected into the right iliac crest at an injection volume of 0.1 ml using a 18G spinal needle.

2) Collection of Bone Marrow Aspirate

24 hours after the injection, 4 ml of a bone marrow aspirate was isolated from the bone marrow, two-fold diluted in PBS, and then carefully placed on Ficoll (GE Health care) contained in a tube (Ficoll: bone marrow aspirate=1:1 v/v). Then, the cell dilution was centrifuged at 2200 rpm for 25 minutes. As a result, a red blood cell layer, a ficoll layer, a mononuclear cell layer and a plasma layer, from bottom to top, were formed. After removing the plasma layer, only the mononuclear cell layer was separated and transferred into a new tube. 30 ml of PBS was added to the obtained mononuclear cell layer and centrifuged again (washing step).

3) Selective Culture of EPCs

The isolated cells were seeded on a 100-mm dish (Falcon) coated with fibronectin and were cultured in EGM-2 (Endothelial growth medium-2, Lonza) for 1 week (5% CO₂ and 37° C.). After the culture, the CFU of the cells was measured and compared with those of the group injected with PBS and the group injected with the positive control G-CSF. The increase (fold) in CFU was compared between the groups, and the results are shown in FIG. 7.

The amount of mobilized EPCs in the group administered with G-CSF known as an EPC mobilization factor was about 4-fold increased compared to that in the negative control group, whereas the amount of mobilized EPCs in the group administered with the composition SP of the present invention was about 10-12 fold increased compared to that in the negative control group and was about 3-fold increased compared to that in the group administered with the positive control G-CSF.

(2) Examination of EPCs in Peripheral Blood after Intra-Bone Marrow of SP

1) Intra-Bone Marrow Administration

Ketamine hydrochloride (50 mg/ml Yuhan, Korea) and Rompun (xylazine hydrochloride, 23.32 mg/ml Bayer Korea) were mixed at a ratio of 2:1, and then 900 μl of the mixture (600 μl of ketamine hydrochloride and 300 μl of Rompun) was injected intramuscularly into New Zealand white rabbits (male, 2.5 kg, n=4). 10 minutes after the intramuscular injection, the same anesthetic (200 μl of ketamine hydrochloride and 100 μl of Rompun) was injected intravenously into the ear of each rabbit to completely anesthetize the rabbits.

SP (Sigma, 4 nmole/kg in saline) dissolved in physiological saline solution was injected into the right iliac crest of one rabbit group at an injection volume of 0.1 ml using a 18G spinal needle.

As a negative control, 1×PBS was injected into the left iliac crest of another rabbit group at an injection volume of 0.1 ml using a 18G spinal needle.

2) Collection of Peripheral Blood

After 3 days after the first injection, 10 ml of peripheral blood was collected. 10 ml of the collected blood was two-fold diluted in PBS and carefully placed on Ficoll contained in a tube (Ficoll: peripheral blood=1:1 v/v). Then, the cell dispersion is centrifuged at 2200 rpm for 25 minutes. The uppermost plasma layer was removed, and only the mononuclear cell layer was separated and transferred into a new tube. 30 ml of PBS was added to the obtained mononuclear cells layer and centrifuged at 1500 rpm for 5 minutes (washing step).

3) Selective Culture of EPCs

The isolated cells were suspended in EGM-2 (endothelial growth medium-2, Lonza). The suspended cells were seeded on a 100-mm dish (Falcon) coated with fibronectin and were cultured in EGM-2 (endothelial growth medium-2, Lonza) for 2 weeks (5% CO₂ and 37° C.).

### Comparative Test Example 1

(1) Comparison of Cell Profiles in Peripheral Blood after Intravenous Administration of SP and G-CSF

1) Comparison of the Effects of Stimulating EPC Mobilization in Peripheral Blood after Intravenous Administration (Comparison of EPC Colony Formation)

According to the same method described in Example 1-(1), 0.1 ml of SP was administered to one rabbit group (New Zealand white rabbits, male, 2.5 kg, n=3), and 0.1 ml of G-CSF (CJ Pharma, Leukokine inj. 150; 2.5 μg/kg) as a positive control was administered to another rabbit group (New Zealand white rabbits, male, 2.5 kg, n=3). 1, 2 and 3 days after the administration, 5-15 ml of peripheral blood was isolated from each animal group. The isolated blood was two-fold diluted in PBS and carefully placed on Ficoll contained in a tube (Ficoll: peripheral blood=1:1 v/v). Then, the cell dilution was centrifuged at 2200 rpm for 25 minutes. The uppermost plasma layer was removed, and only the mononuclear cell layer was separated from the tube and transferred into a new tube. 30 ml of PBS was added to the obtained mononuclear cell layer and centrifuged at 1500 rpm for 5 minutes (washing step). The cells were suspended in EGM-2 (endothelial growth medium-2, Lonza), and the suspended cells were seeded on a 100-mm dish (Falcon) coated with fibronectin and were cultured in EGM-2 (endothelial growth medium-2, Lonza) for 2 weeks (5% CO₂ and 37° C.). After the culture, the produced colonies were stained with toluidine blue and counted.

The results are shown in FIG. 8. As can be seen therein, in the case of intravenous administration of G-CSF, at the first day, EPCs in the peripheral blood were temporarily increased, and after the second day, no EPCs were found in the peripheral blood. On the other hand, in the case of intravenous administration of SP, the amount of EPCs in the peripheral blood was continuously increased. Thus, it could be found that, unlike G-CSF, SP, when administered intravenously, exhibited an excellent effect of stimulating EPC mobilization to peripheral blood over a long period of time.

2) Comparison of Complete Blood Cell Count (CBC) of Peripheral Blood After Intravenous Administration

1 ml of each of the peripheral bloods obtained 1, 2 and 3 days after intravenous administration of SP and G-CSF was placed in an EDTA-containing vial and mixed well to prevent clotting. In the experiment for isolating EPCs from blood cells, the complete blood cell counting of each blood was performed by Royal ARC (Korea) under the same conditions for each blood. The results are shown in FIG. 9.

As can be seen in FIG. 9, G-CSF significantly increased inflammatory cells (neutrophils and monocytes), but SP showed an inflammatory cell profile similar to that of the control group administered with PBS. This suggests that SP did not induce the mobilization of inflammatory cells. Thus, it was confirmed that SP specifically mobilizes only EPCs without causing side effects such as inflammation.

(2) Comparison of Cell Profiles in Peripheral Blood after Intra-Bone Marrow Administration of SP and G-CSF

1) Comparison of the Effects of Stimulating EPC Mobilization after Intra-Bone Marrow Administration (Comparison of EPC Colony Formation)

According to the same method described in Example 2-(2), 0.1 ml of SP was administered to one rabbit group (New Zealand white rabbits, male, 2.5 kg, n=3), and 0.1 ml of G-CSF (CJ Pharma, Leukokine inj. 150; 2.5 μg/kg) as a positive control was administered to another rabbit group (New Zealand white rabbits, male, 2.5 kg, n=3). 1, 2 and 3 days after the administration, 5-15 ml of peripheral blood was isolated from each animal group. The isolated blood was two-fold diluted in PBS and carefully placed on Ficoll contained in a tube (Ficoll: peripheral blood=1:1 v/v). Then, the cell dilution was centrifuged at 2200 rpm for 25 minutes. The uppermost plasma layer was removed, and only the mononuclear cell layer was separated from the tube and transferred into a new tube. 30 ml of PBS was added to the obtained mononuclear cell layer and centrifuged at 1500 rpm for 5 minutes (washing step). The cells were suspended in EGM-2 (endothelial growth medium-2, Lonza), and the suspended cells were seeded on a 100-mm dish (Falcon) coated with fibronectin and were cultured in EGM-2 (endothelial growth medium-2, Lonza) for 2 weeks (5% CO₂ and 37° C.). After the culture, the produced colonies were stained with toluidine blue and counted.

The cell count results are shown in FIG. 10. As can be seen therein,

when SP is administered directly into the bone marrow, it exhibited an excellent effect of stimulating EPC mobilization compared to G-CSF.

2) Comparison of Complete Blood Cell Count (CBC) of Peripheral Blood After Intra-Bone Marrow Administration

1 ml of each of the peripheral bloods obtained 1, 2 and 3 days after intravenous administration of SP and G-CSF was placed in an EDTA-containing vial and mixed well to prevent clotting. In the experiment for isolating EPCs from blood cells, the complete blood cell counting of each blood was performed by Royal ARC (Korea) under the same conditions for each blood. The count results are shown in FIG. 11.

As can be seen in FIG. 11, G-CSF significantly increased inflammatory cells (neutrophils, eosinophils and monocytes), but SP specifically mobilized only EPCs without causing side effects such as inflammation.

1. An agent for stimulating mobilization of endothelial precursor cells, which comprises substance-P in an amount effective for inducing mobilization of endothelial precursor cells from bone marrow.

2. The agent of claim 1, wherein the mobilization of endothelial progenitor cells is mobilization from bone marrow to blood.

3. The agent of claim 1, wherein the mobilization of endothelial progenitor cells is mobilization from bone marrow to injured blood vessels.

4. The agent of claim 3, wherein the endothelial progenitor cells mobilize from the bone marrow to the injured blood vessels and participate in vasculogenesis.

5. The agent of claim 1, which is for intra-bone marrow administration, intravenous administration, subcutaneous administration or intraperitoneal administration.

6. A method for stimulating mobilization of endothelial progenitor cells, comprising administering to a subject substance-P in an amount effective for inducing mobilization of endothelial progenitor cells from bone marrow.

7. The method of claim 6, wherein the mobilization of endothelial progenitor cells is mobilization from bone marrow to blood.

8. The method of claim 6, wherein the mobilization of endothelial progenitor cells is mobilization from bone marrow to injured blood vessels.

9. The method of claim 6, wherein the endothelial progenitor cells mobilize from bone marrow to injured blood vessels and participate in vasculogenesis.

10. The method of claim 6, wherein the subject has ischemic vascular injury or traumatic vascular injury.

11. The method of claim 6, wherein the subject has at least one disease or condition selected from among myocardial infarction, angina, ischemic stroke, cerebrovascular dementia, cerebral infarction, sequelae of cerebral injury, spinal cord injury, sequelae of spinal nerve injury, degenerative diseases, sequelae of cerebral infarction, peripheral nerve disorders, presbyopia, degenerative hearing loss, sequelae of brain surgery, and diabetic ulcer.

12. The method of claim 6, wherein substance-P is administered intra-bone marrow, intravenously, subcutaneously, or intraperitoneally.

13.-15. (canceled)

16. A method for stimulating vasculogenesis in an injured vascular tissue of a subject, the method comprising the steps of:
(a) administering to the subject substance-P in an amount effective for inducing mobilization of endothelial progenitor cells from bone marrow, thereby stimulating mobilization of endothelial progenitor cells from bone marrow to blood;
(b) collecting endothelial progenitor cells from the blood; and
(c) introducing the collected endothelial progenitor cells into the subject.

17. The method of claim 16, further comprising a step of culturing the endothelial progenitor cells collected in step (b), prior to introducing the endothelial progenitor cells into the subject.

18. The method of claim 16, wherein substance-P is administered intra-bone marrow, intravenously, subcutaneously, or intraperitoneally.
